(19) United States
(12) Patent Application Publication
(10) Pub. No.: US 2024/0041553 A1
(43) Pub. Date: Feb. 8, 2024

(54) APPARATUS FOR SETTING A PENETRATION DEPTH OF A TUBULAR OR ROD-SHAPED SLIDING PART IN A RECEIVING PART AND MEDICAL HANDPIECE WITH SUCH APPARATUSES

(71) Applicant: Medi-Globe GmbH, Rohrdorf OT Achenmuehle (DE)

(72) Inventor: Martin STEINIGEWEG, Stephanskirchen (DE)

(73) Assignee: Medi-Globe GmbH, Rohrdorf OT Achenmuehle (DE)

(21) Appl. No.: 18/230,280

(22) Filed: Aug. 4, 2023

(30) Foreign Application Priority Data
Aug. 5, 2022 (EP) 22 188 980.1

(51) Int. Cl. A61B 90/00

(52) U.S. Cl. CPC A61B 90/06; A61B 90/08; A61B 2090/062; A61B 2090/0808

(57) ABSTRACT

An apparatus for setting a penetration depth of a sliding part into a receiving part includes a handle piece, which surrounds the sliding part and which is formed for sliding along a longitudinal axis L of the sliding part. The handle piece includes a clamping device with an adjusting element and a rocker element. The handle piece can pivot the respective rocker element into an arresting pose to the sliding part upon rotating the adjusting element into a blocking direction transversely to the longitudinal axis. In the arresting pose, sliding of the handle piece along the longitudinal axis L is blocked. In order to additionally fix the blocking, the handle piece includes a fixing device that has two fixing elements. In the blocking position, the first and second fixing elements exert a retaining force for fixing the adjusting element against rotation transverse to the longitudinal axis.

### CROSS REFERENCE TO RELATED APPLICATIONS

Applicant claims priority under 35 U.S.C. § 119 of European Application No. 22 188 980.1 filed Aug. 5, 2022, the disclosure of which is incorporated by reference.

### BACKGROUND OF THE INVENTION

### 1. Field of the Invention

The invention relates to an apparatus for setting a penetration depth of a tubular or rod-shaped sliding part, in particular of a medical aspiration needle carrier part, in a receiving part. Thus, the sliding part is adjustable or movable, in particular e.g. slidable, in the receiving part along the longitudinal axis. The invention also relates to a medical handpiece for a medical appliance, with a first and a second apparatus of the above-mentioned type. For example, the handpiece can be included by the medical appliance, and is for example used for endoscopic operations or procedures in the surgery.

### 2. Description of the Related Art

An apparatus and a medical handpiece of the above-mentioned type are known from EP 2 531 122 B1. The apparatus described there is borne under the designation “twistlock”. The apparatus uses a clamping device to position the sliding part in relation to the receiving part in a desired position along the longitudinal axis and to block it there.

The described apparatus includes a handle piece, which surrounds the sliding part. The handle piece is formed for sliding along a longitudinal axis of the sliding part in relation to the sliding part and/or the receiving part. This means, the handle piece and the sliding part and the receiving part, respectively, can be translationally moved to each other. In order to allow sliding, the receiving part and the handle piece can include a receptacle or opening, through which the sliding part is received and can be pushed.

For example, the handle piece can be attached or secured to the receiving part. Thus, the receiving part can be slid together with the handle piece in sliding the handle piece along the longitudinal axis in relation to the sliding part. Alternatively, the handle piece can be formed as a separate element to the receiving part. In sliding along the longitudinal axis of the sliding part, the handle piece can thus also be slid in relation to the receiving part.

The handle piece includes an adjusting element, which is formed for rotating transversely to the longitudinal axis of the sliding part. This means, the adjusting element can be moved around the longitudinal axis, thus in rotational manner. In addition, the handle piece also includes a rocker element, which is arranged between the adjusting element and the sliding part. The rocker element can be slid along the longitudinal axis together with the adjusting element. However, the rocker element is not rotatable in contrast to the adjusting element. Instead, the rocker element is rotationally stationarily or non-rotatably connected to the sliding part. The rocker element and the adjusting element together form the above-mentioned clamping device.

Therein, the adjusting element is formed to pivot the respective rocker element into an arresting pose to the sliding part upon rotating into a blocking direction transversely to the longitudinal axis into a blocking position. In the arresting pose, sliding of the handle piece along the longitudinal axis is blocked. In order to release the blocking, the adjusting element is further formed to pivot the rocker element into a release pose to the sliding part by rotating into an adjusting direction different from the blocking direction transversely to the longitudinal axis into an adjusting position different from the blocking position. In the release pose, sliding of the handle piece along the longitudinal axis is released.

Thus, by rotating the adjusting element, it can be set how deep or far the sliding part can penetrate into the receiving part. Thus, the penetration depth can be particularly simply adjusted or set. If the apparatus is for example employed as an aspiration needle carrier, a protrusion of an aspiration needle from a tube, which is guided in the sliding part, can be preset by the penetration depth. Thus, it can be set by the penetration depth, how far the aspiration needle protrudes out of the tube.

By the apparatus, a single-handed operation and thus the operability of the handpiece for medical personnel is in particular facilitated. In addition, the danger of unintended sliding after effected positioning of the sliding part in relation to the receiving part can be at least largely prevented.

With the twistlock known from the prior art, it can happen in certain situations that the blocking is unintentionally released. For example, this can occur if the apparatus is to be adjusted between multiple different penetration depths for a medical procedure. Then, the handle piece can slip during the procedure, which can for example result in an undesired needle protrusion of the aspiration needle from a tube.

### SUMMARY OF THE INVENTION

It is the object of the present invention to improve handling of the apparatus known from the prior art.

Solutions of the object are specified in the independent claims. Further possibilities of configuration with additional advantages are apparent from the dependent claims, the description as well as the figures.

The invention is based on the realization that for improving the handling of the known twistlock, as it was previously described, an additional latching or locking mechanism can be supplemented. Thus, the point is securing the arresting or blocking. It can be implemented by an additional fixing, securing or locking mechanism of the established twistlock.

In order to realize the additional fixing, the handle piece, as it was initially described, includes at least one fixing device. This means, one or multiple fixing devices can be provided. The respective fixing device includes a first fixing element rotatable to the sliding part transversely to the longitudinal axis by means of the adjusting element. Furthermore, the respective fixing device includes a second fixing element stationary or non-rotatable to the sliding part against the rotation transverse to the longitudinal axis. In the blocking position of the adjusting element, thus in particular if sliding along the longitudinal axis is blocked by means of the rocker element, the respective first and second fixing elements are formed to exert a retaining force for fixing the adjusting element against the rotation transverse to the longitudinal axis.

Otherwise expressed, the first and the second fixing element can be adjusted to each other by or after rotating the adjusting element such that the desired retaining force acts or can act. For example, the adjusting element can be applied with the retaining force. Thereby, the adjusting element can be maintained in the blocking position. By the retaining force, an additional force effort is preferably required to move the adjusting element out of the blocking position, for example into the adjusting position or into another position. Thus, the rotation into the adjusting direction or into the blocking direction can in particular be disabled by means of the retaining force.

Preferably, the retaining force required or sufficient for fixing is preset. This means, the retaining force can have a preset value or range of values. The range of values can for example be ascertained by test experiments. In particular, the retaining force is selected such that the single-handed operation of the apparatus remains further possible.

Hereby, the advantage arises that the apparatus is secured against unintended release of the blocking. After effected positioning of the handle piece in relation to the sliding part and/or receiving part, thus, an unintended sliding of the handle piece can be even more effectively prevented.

The mentioned adjusting element can have at least two different positions or poses. A position can be the above-mentioned adjusting position. In the adjusting position, the handle piece can be in an initial pose. Therein, the handle piece is translationally slidable. In particular, the handle piece is not blocked or arrested and not locked or fixed. The at least one other position can be the above-mentioned blocking position. In the blocking position, the handle piece can be in a zero pose. In the blocking position, the handle piece is blocked and fixed against axial or translational sliding. In particular, the handle piece is not translationally slidable.

Fixing is effected by means of the fixing elements, as they were previously described. For example, the fixing elements can form a complementary fixing structure. This means, the fixing elements can for example form a form- and/or force-fit connection to maintain the blocking position. For example, it can exemplarily be a latching connection or magnetic connection. Thus, the fixing elements can cooperate to provide the retaining force. For example, the first fixing element can be included by the adjusting element. The second fixing element can for example be included by the sliding part and/or the rocker element.

For releasing the fixing and/or blocking, this retaining force has to be overcome. This means, an additional operating force is to be applied in operating the adjusting element to cancel the fixing and in particular also the blocking such that the handle piece is for example again translationally slidable.

The apparatus, in particular the handle piece, can include further aspects or features, as they are already known from the initially described prior art, in particular EP 2 531 122 B1. For example, the rocker element or the sliding part can include respectively associated abutment surfaces, which comprise a friction lining or brake lining. Thus, the blocking or utilization of friction between the rocker element and the sliding part can be achieved. Alternatively, the rocker element and the sliding part can form a respective tooth device, by means of which respective tooth elements engage with associated tooth element receptacles in the arresting pose.

The respective rocker element can for example be arranged in a bearing groove of the sliding part extending along the longitudinal axis by means of a bearing stud. Thereby, the respective rocker element can be non-rotatably (stationarily) guided.

The adjusting element can for example include an oval-symmetrically or crescent shaped opening area. The opening area can be an internal area of the adjusting element receiving the rocker element. Thereby, pivoting of the rocker element can be effected.

Furthermore, the sliding part can for example be formed as a scale piston. This means, the respective sliding part can contain one or more markings (scale) or scale gradations.

In the following, embodiments of the invention are described, by which further advantages arise.

In the following embodiments, the point is first how the fixing, thus the locking mechanism, can be realized. For example, the fixing device can be conceived by means of an active or a passive or a combination of an active and passive fixing mechanism.

According to an embodiment of the invention, the active fixing can for example be implemented in that the first fixing element forms a barrier body and the second fixing element forms a notch or recess for receiving the barrier body. The barrier body is formed to be moved or adjusted into a fixing pose in the blocking position of the adjusting element by means of an operating action of an operator. In the fixing pose, the retaining force is maximum.

For releasing the fixing, thus for unlocking, it can be provided analogously thereto that the barrier body is formed, in the fixing pose, to be moved or adjusted into an unlocking pose by means of an operating action of an operator. In the unlocking pose, the retaining force is minimum, in particular reduced, preferably equal to zero (nullified).

This means, a manual operation in addition to the rotation is required to fix or unlock the adjusting element. By means of the operating action, the first fixing element can be adjusted or moved between at least two poses. One pose is the fixing pose. A further one of the poses is the unlocking pose.

In the fixing pose, the barrier body can be connected to the notch in form-fit manner for example by means of latching connection or plug connection. This means, the barrier body is received in the notch or inserted into it.

The barrier body can for example be formed as a knob or pin. In addition, the barrier body can for example include one or more spring elements to allow easier release of the fixing.

In a specific configuration, a pin or knob (barrier body) to be triggered by manual actuation can be incorporated in the twistlock rotation mechanism (adjusting element). For example, it can be internally equipped with a coil spring. For arresting, the twistlock rotation mechanism is rotated, as previously described. For securing the arrest, the pin or knob is downwards pressed by additional, manual operating action. An internal contour of the pin or of the knob engages with the recess (notch) provided thereto, which can for example be incorporated in the sliding part. For releasing the fixing and the arrest, thus, the pin or knob has to be again pressed or pulled (push-push or push-pull operation) before the rotational movement of the twistlock, in order that it is removed from the provided recess. Only then, the possibility of rotation, thus, the rotation of the twistlock, is again released. Overall, the active fixing can thus be implemented.

Implementation examples for the above mentioned passive fixing are specified in the following embodiments.

According to an embodiment, the passive fixing can be realized in that the adjusting element is formed to move the first fixing element into a fixing pose to the second fixing element, in which the retaining force is maximum, by rotating transversely to the longitudinal axis into the blocking position. Thus, the fixing is automatically effected by rotating the adjusting element. Thereby, the fixing is an additional effect, which can be brought about upon blocking.

For releasing the fixing, thus for unlocking, a corresponding passive unlocking can be provided analogously thereto. This means, the adjusting element can be formed to move the first fixing element into an unlocking pose to the second fixing element, in which the retaining force is minimum or nullified, by rotating transversely to the longitudinal axis for example into the adjusting position or a position different from the blocking position.

By means of rotation of the adjusting element, the fixing elements can thus be adjusted between the previously described at least two poses, namely the fixing pose and the unlocking pose.

According to a further embodiment, the first and the second fixing element can include a magnetic material and form a magnetic field of opposite polarity to each other. Thus, the fixing can be effected by force fit, in particular a magnetic connection, of the fixing elements. In the fixing pose, an attraction force or magnetic force of the fixing elements can be as great as the fixing elements attract each other. In the unlocking pose, in contrast, attraction is not effected. The fixing elements can form magnets, in particular permanent magnets.

Specifically, the magnetic fixing could for example be realized by magnets included by the adjusting element and a magnet of opposite polarity included by the scale piston or the rocker element. In particular, a magnetic rail can for example be incorporated in the sliding part, which extends over the entire length of the sliding part along the longitudinal axis. The magnetic rail can for example be mounted or fixed in the volume or on the surface of the scale piston. Similarly, a magnet of opposite polarity can be incorporated by the twistlock rotation mechanism, thus be embedded on the surface or in the volume. Structurally, thus with respect to the position, the magnet associated with the twistlock is positioned with great distance, in particular rotationally viewed spaced from the magnetic rail in the scale piston, in the opened state (adjusting position). By the rotational movement of the twistlock into the blocking position, the position of the magnet is manipulated such that it is congruent over the magnetic rail of the scale piston. Thus, the maximum magnetic force can form. Thereby, a mechanic blocking of the twistlock can be supported and an unintended release of the twistlock can be prevented.

According to a further embodiment, the passive locking is realized in that the first fixing element is formed as a rolling bearing with at least one rolling body. The second fixing element is formed as a notch for receiving the rolling body in the fixing pose. In the fixing pose, the rolling body can thus be arranged or attached in the notch. A form-fit connection can be realized.

The rolling bearing can for example be a ball bearing known per se. Correspondingly, the rolling body can for example form a ball. The rolling body can move along the surface of the sliding part, preferably substantially without friction, upon rotating the adjusting element. In order to move the rolling body into the notch, the rolling body can for example be spring-loaded. The notch or recess can be a depression in the sliding part formed complementarily to the rolling body, into which the rolling body can latch or slide.

In a specific implementation, the twistlock can for example include one or more ball bearings, in particular of metal, which plunge into geometric pockets (notch) of the scale piston and thereby make additional forces for releasing the twistlock rotation mechanism necessary. Thereto, individual or multiple ball bearings can for example be incorporated or embedded in the inner side of the adjusting element facing the scale piston. In the released state of the twistlock rotation mechanism (adjusting position), the respective ball bearing can rest on the closed surface of the scale piston. Upon rotating the twistlock, the balls of the ball bearing can preferably roll or rotate on the surface without friction. In the blocking position, thus upon arresting the twistlock by means of the rotation, the balls of the ball bearings can drop into or engage with pockets or recesses correspondingly embedded in the surface of the scale piston. Thereby, the additional locking is realized. For releasing the arrest, an additional release mechanism, thus an additional operating action, is not required. For example, it can be sufficient to rotate the adjusting element with increased force effort to overcome the form fit. Thereto, the respective notch can for example be adapted to the geometry of the respective rolling body, thus for example the ball.

As previously mentioned, the handle piece can for example include one or more of the fixing devices of the previously described types. For example, the handle piece can comprise multiple, thus two or more, similar fixing devices. With similar, it is meant that the fixing devices comprise the same fixing elements and thus realize a same fixing mechanism, as it was previously described. Alternatively, the handle piece can for example include multiple, thus two or more, different fixing devices. Thus, the fixing devices can comprise fixing elements different from each other and realize different fixing mechanisms, as they were previously described.

For example, a combination of a passive and active locking can be implemented. For example, it can be provided that the fixing is passively effected, while unlocking is actively performed. Thus, the fixing elements can be automatically moved into the fixing pose by rotating the adjusting element transversely to the longitudinal axis into the blocking position, as described for the passive locking. In order to release the fixing, in contrast, the additional operating action, as it was described for the active locking, is required in the blocking position of the adjusting element to move the fixing elements from the fixing pose into the unlocking pose. Specifically, it could for example be provided that upon locking, a locking body, thus for example an above-mentioned rolling body, drops into a provided pocket (notch) of the scale piston, but which has to be actively released for releasing. This means, the locking body for example has to be intentionally pulled up, slid or pressed.

According to a further embodiment, the adjusting element and the rocker element form a guide structure to each other, in particular in relation to each other. The guide structure presets a guide course at least for rotating the adjusting element transversely to the longitudinal axis in relation to the rocker element. The adjusting element is adjustable along the guide course into the respective position, thus for example the above mentioned adjusting and blocking position or further positions described in more detail later.

Thus, the guide course or the guide structure presets a direction of movement or trajectory for the adjusting element. This discrete guide course can allow a particularly simple operation by the operator.

The guide structure can for example be formed by two guide bodies. The guide bodies can be coupled to each other, in particular connected to each other in form-fit manner. The first guide body can for example be a knob or a pin or a stud. The second guide body can for example be a guide groove or guide rail. The first guide body can engage with the second guide body. The second guide body can preset the guide course. The guide bodies can be movable in relation to each other. The first guide body can for example be formed non-rotatably to the sliding part. For example, the first guide body can be included by the sliding part or the respective rocker element. The second guide body can be formed rotatably to the sliding part. For example, the second guide body can be included by the adjusting element.

In this context, it is provided in a further embodiment that the guide structure includes a guide course section associated with the blocking position, which extends along the longitudinal axis. For releasing the fixing or the blocking, the adjusting element is slidable along the guide course section in relation to the respective rocker element.

This means, by the guide course, a possibility of particularly securely fixing the handle piece can be provided. Therein, an additional operating movement, in particular sliding of the adjusting element, is required for unlocking to release the fixing. In particular, the unlocking can be initiated by sliding the adjusting element out of the blocking position along the longitudinal axis. The sliding movement has to be intentionally performed, whereby an indication or warning function for the operator is realized.

Thereto, the guide course in the blocking position is configured transversely to the blocking direction offset along the longitudinal axis. Thus, a step-shaped or staircase-shaped course profile of the guide structure, in particular of the second guide body, arises.

Alternatively thereto, a particularly simple unlocking mechanism can be realized in that for unlocking the adjusting element is for example formed for rotating out of the blocking position into the adjusting direction. This means, the adjusting element can be directly brought or moved from the blocking position into the adjusting position by rotating into the adjusting direction. Thereto, the guide structure can for example preset a rectilinear guide course.

Additionally or alternatively to the guide structure, a further unlocking mechanism can be provided, which implements a particularly secure fixing and a particularly intentional unlocking.

Thereto, the adjusting element is formed for releasing the fixing according to a further embodiment to occupy an unlocking position different from the blocking position and the adjusting position by rotating from the blocking position into the blocking direction. Thereby, the adjusting element is formed to move the first and the second fixing element into the above-mentioned unlocking pose, in which the retaining force is reduced, preferably minimum, compared to the fixing pose. However, the respective rocker element maintains the arresting pose in the unlocking position of the adjusting element.

This means, in the unlocking position, the handle piece is further blocked against sliding along the longitudinal axis. Thus, the handle piece is not adjustable. However, the handle piece is no longer locked or fixed. This means, the rotation of the adjusting element is again possible without additional force effort.

For unlocking, the adjusting element thus is to be rotated into the same direction as for blocking, namely into the blocking direction and not into the adjusting direction. Thereby, an additional intentional operating action is required to release the fixing and subsequently also the blocking. Thus, a particularly secure blocking and fixing of the handle piece against sliding arises.

In this context, it is provided according to a further embodiment that the handle piece includes an unlocking device. The unlocking device is arranged between the sliding part and the adjusting element. The unlocking device is formed to maintain the respective fixing elements in the unlocking pose by or in rotating the adjusting element transversely to the longitudinal axis.

Preferably, the unlocking device can maintain the respective fixing elements in the unlocking pose only if the adjusting element is outside of the blocking position. Thus, the fixing can be prevented outside of the blocking position. Thereto, the fixing elements can for example be axially (along the longitudinal axis) maintained or arranged at a preset distance to each other by means of the unlocking device.

In the following embodiments, it is addressed in more detail how the unlocking device can be specifically realized.

According to an embodiment, the unlocking device includes a first unlocking element. The first unlocking device is arranged stationarily, thus non-rotatably, against the rotation transverse to the longitudinal axis to the sliding part. Thus, the adjusting element can be rotationally rotatable in relation to the first unlocking element.

The adjusting element and the first unlocking element form a spacing structure. The spacing structure is formed to maintain the adjusting element and the first unlocking element at a preset distance to each other along the longitudinal axis in relation to each other upon rotating the adjusting element transversely to the longitudinal axis, thus in particular outside of the blocking position. Thereby, the unlocking pose of the fixing elements is realized. This means, the distance can be transferred to the fixing elements.

In addition, the spacing structure includes a connection mechanism, by means of which the first unlocking element and the adjusting element are connected to each other in form-fit manner in the blocking position and therein the preset distance is reduced. By the form-fit connection, the fixing pose of the fixing elements is realized.

Thus, the adjusting element and the first unlocking element can be translationally slidable to each other. By the distance, it is presently meant that the adjusting element and the first unlocking element are retained slid, in particular pulled apart, in relation to each other along the longitudinal axis outside of the blocking position. Otherwise expressed, the fixing and the unlocking can be realized in that the adjusting element and the first unlocking element are slid, in particular pulled apart, in relation to each other along the longitudinal axis. Therein, the first unlocking element can be further moved towards the edge of the overlap area or out of the overlap area at least in certain areas with the adjusting element in the overlap area. Thus, a visible gap between adjusting element and first unlocking element does not have to arise. By sliding, the fixing elements can be pulled apart (unlocking pose) or connected (fixing pose).

The spacing structure can be formed by two complementary spacing bodies, one of which is included by the adjusting element and the other one by the unlocking element. For example, the spacing bodies can form a supporting part and a retaining part. Outside of the blocking position, they can be supported against each other. In the blocking position, the supporting part can engage with the retaining part, such that the form fit forms. The supporting part can for example be formed as a stud or projection or nose. The retaining part can form a resting surface for the supporting part, which extends transversely to the longitudinal axis. Upon rotating, the retaining part can slide along the resting surface. The resting surface can include a groove or receptacle corresponding with the blocking position, into which the retaining part can be fitted. Thereby, the connection mechanism can be realized. The connection mechanism can allow an additional fixing to the fixing elements.

The geometric shape of stud and groove can be selected such that the rotation is blocked. Thus, an additional operating action such as for example pulling apart can be required to release the form-fit connection. A corresponding movement course can for example be preset by the guide course section of the guide structure extending parallel to the longitudinal axis.

According to a further embodiment, the unlocking device includes a signaling element, which protrudes beyond the adjusting element at least in certain areas along the longitudinal axis for signaling the blocking and fixing only in the blocking position by sliding the adjusting element and the first unlocking element.

Thus, the signaling element is in particular only visible in the blocking position. Outside of the blocking position, the signaling element is preferably completely covered or overlapped by the adjusting element and/or the first unlocking element.

Thereto, the signaling element can be arranged rotationally and translationally stationarily to the sliding part in the blocked state of the handle piece. For example, the signaling element can include a signal color, such as for example red. Thus, the blocking and/or fixing can be particularly uniquely indicated. The signaling element can for example form a stop, thus an abutment area, for the receiving part.

According to a further embodiment, the unlocking device comprises a second unlocking element. The second unlocking element is stationarily arranged against sliding along the longitudinal axis in relation to the adjusting element. This means, the second unlocking element is in particular only slidable together with the adjusting element. The first and the second unlocking element form a sliding structure. The sliding structure is formed to slide the adjusting element and the first unlocking element along the longitudinal axis in relation to each other into the preset distance to each other upon rotating the adjusting element transversely to the longitudinal axis out of the blocking position into the unlocking position.

This means, the second unlocking element can be used to release the form fit of the connection mechanism of the spacing structure of the adjusting element and the first unlocking element.

The sliding structure can be formed by two complementary sliding bodes, one of which is included by the first and the other one by the second unlocking element. For example, the sliding bodies can form a supporting part and a retaining part analogously to the above-mentioned spacing bodies. Outside of the blocking position, they can be supported against each other. In the blocking position, the supporting part can engage with the retaining part such that a form fit forms. The supporting part can for example be formed as a stud or projection or nose. The retaining part can form a resting surface for the supporting part, which extends transversely to the longitudinal axis. Upon rotating, the retaining part can slide along the resting surface. The resting surface can include a groove or receptacle corresponding with the blocking position, into which the retaining part can be fitted.

Preferably, the geometric shape of stud and groove is selected such that sliding is automatically effected by rotating the adjusting element, preferably only upon rotating into the blocking direction. Thereto, the sliding bodies can include a respective abutment surface, by means of which the sliding bodies abut on each other in the blocking position, which is oblique transversely to the longitudinal axis. An inclined plane or ramp arises. Thereby, the sliding bodies can particularly simply slide along each other upon rotating the adjusting element. Thereby, the adjusting element and the first unlocking element are moved apart until the desired distance is achieved.

According to a further embodiment, the adjusting element and the second unlocking element include a coupling structure for carrying the second unlocking element at least in sections upon rotating the adjusting element transversely to the longitudinal axis. Therein, the coupling structure is formed to offset the spacing structure and the sliding structure upon sliding the adjusting element from the unlocking position into the adjusting position transversely to the longitudinal axis and thereby maintain the preset distance upon rotating into the adjusting direction.

By the rotational offset, it can be prevented that the connection mechanism of the adjusting element and the respective unlocking device grip when the adjusting element is rotated back into the initial pose, thus into the adjusting position.

By the coupling structure, the second unlocking element is thus formed to be rotated in relation to the sliding part in the coupled state by rotating the adjusting element transversely to the longitudinal axis. The adjusting element carries along the second unlocking element. In the uncoupled state, in contrast, the second unlocking element is not carried along. It remains non-rotatable to the sliding part.

For example, the coupling structure can include an entraining body and a carrying body, one of which is arranged at the second unlocking element and one at the adjusting element. In a coupling pose, thus in the coupled state, the bodies abut on each other or engage with each other. Therein, the carrying body can apply the entraining body with a force or with a pulse, whereby the rotational movement around the longitudinal axis is realized.

Thus, the unlocking device can also realize a locking function (allowing and/or maintaining the fixing pose) besides the unlocking function (allowing the unlocking pose). Therefore, the unlocking device can also be referred to as locking device. Analogously, the unlocking elements can also be referred to as locking elements.

A further aspect of the invention relates to a medical handpiece for a medical appliance. For example, the handpiece can be included by or associated with the medical appliance. The medical handpiece includes a first and a second apparatus of the above-mentioned type. Therein, the first apparatus is formed to set the penetration depth of a first tubular sliding part in a first receiving part. The second apparatus is formed to set the penetration depth of a second tubular sliding part in a second receiving part. The first and the second apparatus are arranged one behind the other such that the first sliding part of the first apparatus is at least partially formed as a second receiving part for the second sliding part of the second apparatus. The first receiving part and the second sliding part additionally comprise a respective connection interface for connecting elements of the medical appliance.

Thereby, a particularly simply and safely operable handpiece for a medical appliance can be provided. The handpiece can be particularly securely maintained in the respective adjusting position, thus with the desired penetration depth, by the two apparatuses. Thus, irritations for the handling personnel do not occur in the course of adjusting procedures.

Preferably, the second receiving part of the handpiece can be connected to the handle piece of the second apparatus. Correspondingly, the second receiving part and the handle piece can be commonly translationally movable along the longitudinal axis. Hereby, the advantage arises that the length of the handpiece can be adapted to different types of medical appliances, in particular to endoscopes. Thus, the handpiece is universally employable.

In contrast thereto, the first receiving part can for example be formed separately from the handle piece of the first apparatus. Thus, the handle piece can be translationally moved in longitudinal direction, thus along the longitudinal axis, in relation to the first receiving part and the first sliding part. Thus, the handle piece can be used as a stopper for the first receiving part.

The handpiece can include further aspects and features as they have already been described in the prior art, in particular EP 2 531 122 B1.

For example, the first receiving part can include a joining element for connecting or joining a medical hollow or aspiration needle. Therein, the hollow or aspiration needle can be wrapped by the medical handpiece in the connected state. This means, the needle can extend through the first receiving part, the first sliding part and the second receiving part adjoining thereto and the second sliding part. In particular, the first receiving part can then be formed to preset the projection of the aspiration needle from a tube by sliding along the longitudinal axis. Thereto, the first receiving part can for example be formed for translationally sliding separately from the handle piece of the second apparatus.

The second sliding part can for example also comprise a connecting element for connecting or joining or coupling to a connecting element of a further medical appliance, in particular a working channel of an endoscope, at its distal end. At the proximal end area of the first receiving part, a connecting element can also be arranged. Via this connecting element, a further medical appliance, such as for example a syringe or a suction device or a flushing device, can for example be joined.

Preferably, the receiving part can be in an initial pose as a handle part. This facilitates the operability of the handle piece in adjusting the two sliding parts.

The invention also includes the combinations of the described embodiments. In addition, developments of the handpiece according to the invention are also encompassed by the invention, which comprise features, as they have already been described in context of the developments of the apparatus according to the invention. For this reason, the corresponding developments of the handpiece according to the invention are not again described here.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1 is a schematic representation of an apparatus for setting a penetration depth of a tubular or rod-shaped sliding part in a receiving part according to an exemplary embodiment;

FIG. 2 is a schematic representation of the apparatus with a handle piece, which is in an initial state;

FIG. 3i is a schematic representation of the apparatus, in which the handle piece is in a zero state;

FIG. 4 is a schematic representation of the apparatus with the handle piece in a first pose when bringing the handle piece from the zero state into an unlocking state;

FIG. 5 is a schematic representation of the apparatus with the handle piece in a second pose when bringing the handle piece from the zero state into an unlocking state;

FIG. 6 is a schematic representation of the apparatus, in which the handle piece is in the unlocking state;

FIG. 7 is a schematic representation of the apparatus with the handle piece in a first pose when bringing the handle piece from the unlocking state into the initial state; and

FIG. 8 is a schematic representation of the apparatus, in which the handle piece is again in the initial state.

### DETAILED DESCRIPTION OF PREFERRED EMBODIMENTS

The embodiments explained in more detail in the following are to be regarded as preferred embodiments of the invention. The components of the embodiments described in the embodiments each represent individual features of the invention to be considered independently of each other. These features also each develop the invention independently of each other. Therefore, they are also to be regarded as constituents of the invention in individual manner or in a combination different from the shown one. In addition, further ones of the already previously described features of the invention can supplement the described embodiments.

For functionally identical elements, the same reference characters were respectively assigned in the figures.

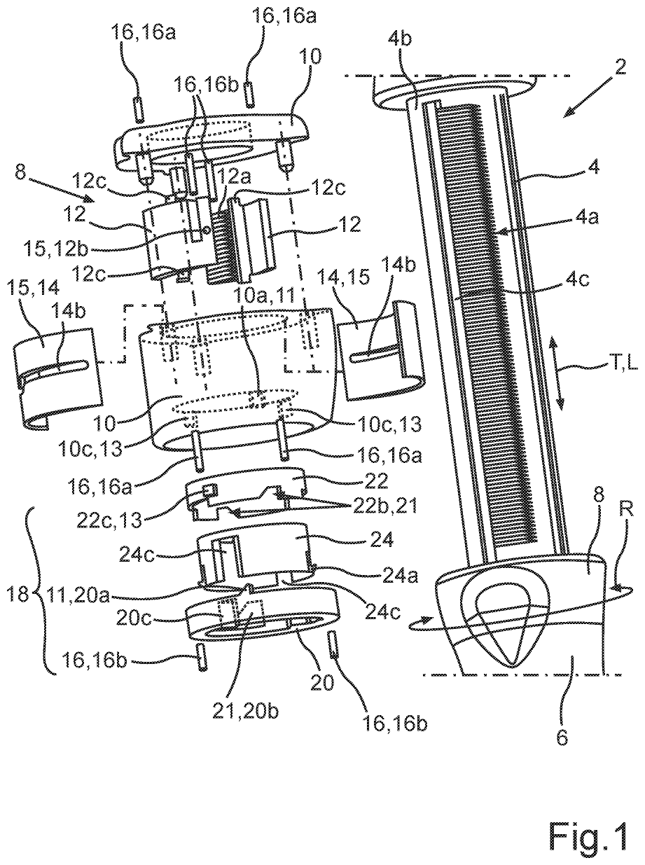

FIG. 1 shows a schematic representation of an apparatus 2. The apparatus is employed for setting a penetration depth of a tubular or rod-shaped sliding part 4 in a receiving part 6. Thereto, the apparatus 2 includes a handle piece 8, which surrounds the sliding part 4, besides the sliding part 4 and the receiving part 6. The handle piece 8 is formed in relation to the sliding part 4 and/or the receiving part 6 along a longitudinal axis L of the sliding part 4. This means, the handle piece 8 can be translationally slid or moved along the sliding part into the sliding direction T. In order to allow sliding, the receiving part 6 and the handle piece 8 include an opening not illustrated in more detail for receiving and guiding the sliding part 6 along the sliding direction T.

In order to quantify the penetration depth or introduction depth, the sliding part 4 is presently formed as a scale piston. This means, a scale 4b is indicated on the sliding part 4, with measurement values or numerical values, which preset the penetration depth. In the present embodiment, the scale 4b for example indicates numerical values up to 4 centimeters, and is divided into millimeter steps. Thus, the penetration depth can be particularly precisely adjusted.

In order to set the penetration depth, the handle piece 8 includes a blocking mechanism. By the blocking mechanism, the handle piece 8 can be blocked at a desired location along the longitudinal axis L against sliding along the sliding direction. In this pose, the handle piece 8 forms a stop for the sliding part 4 and/or the receiving part 6, in particular a head of the sliding part 4 not illustrated in more detail, and thereby limits the penetration depth.

The blocking mechanism used for blocking is known per se from the prior art, in particular EP 2 531 122 B1. In the following, the blocking mechanism is described in more detail based on a preferred embodiment. However, other known types of the blocking mechanism can of course also be employed. For explaining the blocking mechanism of the handle piece 8, the handle piece 8 is illustrated in an exploded representation in FIG. 1.

In order to realize the blocking, the handle piece 8 includes an adjusting element 10, which is also referred to as twistlock in the following. The adjusting element 10 is formed for rotating transversely to the longitudinal axis L of the sliding part 4. This means, the adjusting element can be moved or rotated around the sliding part into the drawn rotational direction R, thus in rotational manner. As shown in FIG. 1, the adjusting element 10 is presently exemplarily formed two-part. The two parts of the adjusting device 10 can be connected to each other in form-fit manner via predetermined connecting elements. By the two-part shape, the advantage arises that the adjusting element can be particularly simply assembled or composed.

Furthermore, the handle piece 8 includes at least one, presently exemplarily two, rocker elements 12. The rocker elements 12 are arranged along the longitudinal axis L on opposing sides of the sliding part 2 between the adjusting element 10 and the sliding part 4. The rocker elements are slidable together with the adjusting element 10 along the longitudinal axis. In contrast to the adjusting element 10, however, the rocker elements are radially or rotatably stationarily, thus non-rotationally or non-rotatably, connected to the sliding part 4.

Thereto, the sliding part 4 includes a respective bearing groove 4c along the longitudinal axis L and the rocker elements include a respective bearing stud 12c. The respective rocker element 12 is fixed or supported in the associated bearing groove 4c by the bearing stud 12c. Thereby, the handle piece 8 is coupled or connected to the sliding part via the rocker elements 12. The respective bearing groove 4c presets the sliding direction T for the handle piece 8.

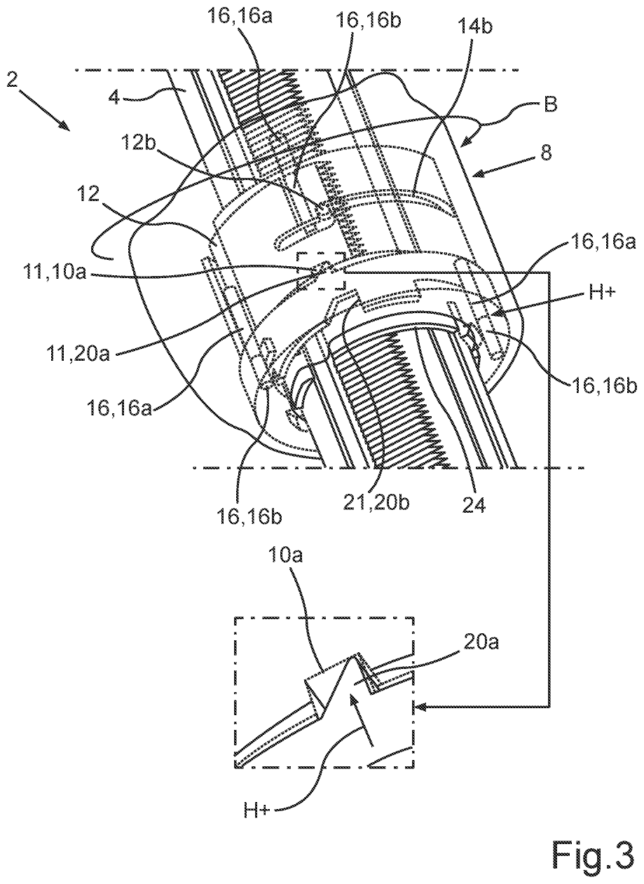

The adjusting element 10 forms a clamping device known per se with the respective rocker element 12. Therein, the adjusting element 10 is formed to pivot the respective rocker element 12 into an arresting pose to the sliding part 4 by rotating into a blocking direction B (presently a clockwise rotation), which extends transversely to the longitudinal axis, into a blocking position. In the arresting pose, sliding of the handle piece 8 along the longitudinal axis is blocked. Thus, the handle piece is arrested and is in a zero state. FIG. 3 exemplarily shows the handle piece in the zero state.

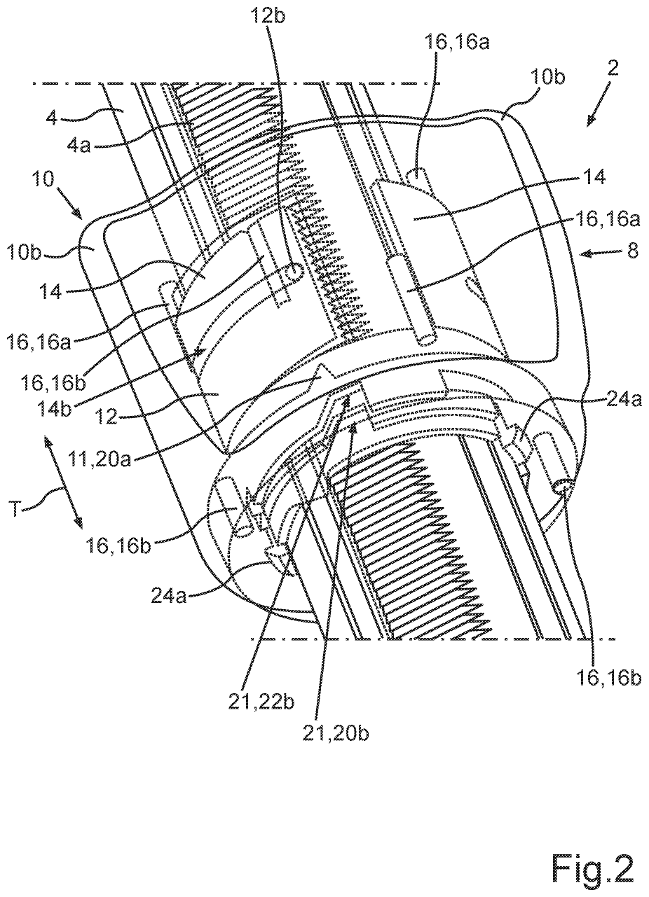

In contrast thereto, the adjusting element 10 is further formed to pivot the respective rocker element 12 into a release pose to the sliding part 4 by rotating into an adjusting direction V different from the blocking direction B transversely to the longitudinal axis L (presently a counterclockwise rotation) into an adjusting position different from the blocking position. In the release pose, sliding of the handle piece 8 along the longitudinal axis L is released, thus no longer blocked. The handle piece is in an initial state, as it is exemplarily shown in FIG. 2.

How the respective rocker element 12 and the adjusting element 10 can be formed to allow the blocking or arresting, is known per se. In the following embodiment, the respective rocker element includes a tooth device 12a on an inner side or resting surface facing the sliding part 4. The sliding part 6 also includes a corresponding tooth device 4a on an outer side or resting surface facing the respective rocker element 12.

Upon rotating the adjusting element 10 into the blocking position, the adjusting element 10 tilts or pivots the respective rocker element 12 along the axis formed by the respective bearing stud or the respective bearing groove 12c, 4c, whereby the tooth devices 4a, 12a are brought into contact or connection with each other. In the arresting pose, the tooth devices 4a, 12a thus abut on each other. Therein, tooth elements engage with tooth element receptacles of the tooth devices 4a, 12a. A form-fit connection forms.

In contrast, upon rotating into the adjusting position of the adjusting element 10, the respective rocker element 12 is pivoted or tilted such that the contact between the tooth devices 4a, 12a is released. The respective rocker element 12 is in the release pose. The blocking is released.

Instead of the tooth devices 4a, 12a, the blocking mechanism can for example be implemented by a brake lining. In the arresting pose, the respective resting surface of the rocker element 12 can thus act like a brake wedge.

The described apparatus 2 has the advantage that it can be particularly simply operated in single-handed manner. Thus, the apparatus 2 is for example suitable in a manner known per se for the employment in a medical handpiece. A corresponding medical handpiece can for example be employed for endoscopic procedures. Therein, one or more, in particular two, of the described apparatuses are assembled or installed to each other in known manner. The resulting handpiece can then be connected to one or more medical appliances such as for example an endoscope and/or a suction device and/or an aspiration needle via provided interfaces. By means of the handpiece, by adjusting the penetration depth of the respective apparatus in an endoscopic procedure, a projection of an aspiration needle part of the aspiration needle from a tube, which is for example passed through the medical handpiece, can for example be set. Thus, a puncture depth for a tumor or another tissue piece can for example be particularly accurately adjusted. A medical handpiece of the designated type is known per se from the prior art and is therefore not described in more detail in the following.

In order to preset a respective movement direction for the adjusting element 10, the handle piece 8 includes at least one, presently for example two, guide structures 15. The respective guide structure 15 includes a first guide body and a second guide body. Presently, the first guide body is exemplarily formed as a guide element 14. The respective guide element 14 is presently for example attached or fixed to an inner side of the adjusting element 10 facing the respective rocker element 12. Each of the guide elements 14 includes a guide channel 14b or a guide groove, by which a discrete guide course for the adjusting element 10 is preset. The guide rail 14b is presently for example formed as a cutout or recess in the guide element 14.

Presently, the respective second guide body is exemplarily formed as a guide pin 12b. The guide pin 12b is included by the respective rocker element 12. The guide pin 12b is fixed to an outer side of the respective rocker element 12 facing the adjusting element 10. Presently, the guide pin 12b forms an elevation or a nose, which extends radially, thus perpendicularly to the longitudinal axis L, towards the guide element 14.

The guide pin 12b and the guide channel 14b are connectable in form-fit manner. Thereto, the guide pin 12b can engage with the guide rail 14b. This means, the guide pin 12b can be arranged in the guide rail 14b. In the arranged state, the guide pin 12b and the guide element 14 are movable in relation to each other, in particular into the rotational direction R and/or the sliding direction T. The movement direction is determined by the course of the guide channel 14b. In that the adjusting element 10 is fixedly connected to the guide element 14, thus, the adjusting element is also only movable along the guide course preset by the guide rail 14b in relation to the rocker element 12 in the arranged state.

In the present embodiment, the guide course has a step shape or Z shape (see FIG. 2 or FIG. 3). This means, the guide course comprises three connected guide course sections. Two of the sections extend along the rotational direction R and are connected by a section, which extends in the sliding direction T. An offset of the guide course in longitudinal direction L, presently in relation to the blocking direction B, for example downwards, arises. The offset is in the blocking position of the adjusting element 10. This means, the sections parallel to the rotational direction R end or begin in the blocking position.

The blocking for setting the penetration depth can unintentionally release in certain situations. In order to avoid such unintended release of the blocking, a fixing mechanism is provided in addition to the blocking mechanism. By the fixing mechanism, the blocking can be fixed or locked. Then, an additional force effort is for example required to be able to release the blocking. With releasing, it is presently in particular meant that the adjusting element is moved from the blocking position into the adjusting position and thus the respective rocker element 12 is moved from the arresting pose into the release pose.

In order to implement the fixing mechanism, the handle piece 8 includes a fixing device 16. Presently, multiple such fixing devices 16, in particular four such fixing devices 16, are for example provided. The respective fixing device 16 includes a respective first fixing element 16a. The respective first fixing element 16a is rotatable by means of the adjusting element 10 to the sliding part 4 transversely to the longitudinal axis L, thus into the rotational direction R. In the present embodiment, the respective first fixing element 16a is included by the adjusting element 10 thereto. The four first fixing elements 16a are attached in the adjusting element 10 equally distributed in the rotational direction R. However, in sliding direction T, two of the fixing elements 16a are therein arranged upwards offset, thus towards the sliding part 4. The remaining two fixing elements 16a are arranged downwards offset, thus towards the receiving part 6.

Furthermore, the respective fixing device 16 includes a second fixing element 16b, which is stationarily or non-rotatably arranged to the sliding part 4. Thereto, the respective second fixing element 16b can for example be integrated in the sliding part 4 itself. Alternatively, the respective second fixing element 16, as shown in the embodiment, can be installed in a component of the handle piece 8 non-rotatable to the sliding part 6. Presently, two of the second fixing elements 16b are exemplarily included by respectively one of the rocker elements 12. The remaining two fixing elements 16b are for example integrated or arranged by an unlocking element 20 of an unlocking device 18 designated in more detail later. The second fixing elements 16b are arranged offset in relation to the rotational direction R and sliding direction T analogously to the first fixing elements 16a.

In the blocking position of the adjusting element 10, the respective first and second fixing elements 16a, 16b are now formed to exert a retaining force H for fixing the adjusting element against rotation transverse to the longitudinal axis L. This means, in the blocking position, the handle piece 8 is blocked and additionally fixed or locked. By the retaining force, the adjusting element 10 can thus no longer be rotated into the rotational direction R. In order to allow the rotation, the retaining force H first has to be overcome. For overcoming the retaining force H, a larger force effort is preferably required than it would be required for releasing the blocking in itself.

In the embodiment in FIG. 1, the respective fixing device 16 is realized as a passive fixing or locking. This means, the respective first fixing element 16a is movable in relation to the second fixing element 16b into a preset fixing pose, in which the retaining force H is maximum, by means of the adjusting element 10 by rotation thereof transverse to the longitudinal axis into the blocking position. Thus, the fixing is automatically realized by rotating the adjusting element.

The first and the second fixing element 16a, 16b are presently formed as permanent magnets. Therein, the respective first and second fixing elements 16a, 16b comprise a magnetic material with a magnetic field of opposite polarity. Thus, the retaining force H is provided by the magnetic force, by means of which the permanent magnets attract each other.

Presently, the permanent magnets have a cylindrical shape. Therein, the structure of the magnets is selected such that the magnetic force thereof acts in sliding direction T. This means, the retaining force H is presently maximum exactly if a respective first fixing element 16a is arranged parallel in a straight line to the associated second fixing element 16b (see for example FIG. 3). Thereby, the offset of the fixing elements 16a, 16b in rotational direction R is presently selected such that the cylindrical magnets of a first and the associated second fixing element 16a, 16b abut on each other in the blocking position with the ends of opposite polarity, preferably are directly in contact with each other. The above mentioned fixing pose arises. Outside of the blocking position, in particular in the adjusting position, in contrast, the associated fixing elements 16a, 16b are offset to each other or spaced in sliding direction T on the one hand and in adjusting direction R on the other hand. This pose of the fixing elements 16a, 16b is also referred to as unlocking pose in the following.

Alternatively to the embodiment in FIG. 1, the fixing elements 16a, 16b can for example be formed as rolling bearings with at least one rolling body and a notch for receiving the rolling body in the fixing pose. Alternatively, an active fixing is for example of course also conceivable. Therein, the fixing elements 16a, 16b can for example be formed as barrier body and notch for receiving the barrier body. For moving into the fixing pose, an operating action of an operator provided in addition to the rotation is required.

Analogously to the fixing, an active or passive unlocking, thus passive or active release of the fixing, can be provided. A passive unlocking can for example be realized in that the adjusting element 10 is rotated out of the blocking position into the adjusting direction V. If the rotation is effected with a sufficiently great force effort, the retaining force H of the fixing device 16 can be released. Therein, the fixing element 16a, 16b can be moved into the unlocking pose, in which the retaining force H is at least reduced, in particular minimum or nullified. By rotating into the adjusting direction, however, blocking of the respective rocker element 12 is additionally also released, since the respective rocker element 12 is moved from the arresting pose into the release pose.

In order to make the fixing even more secure, and to provide a very intentional unlocking, the active unlocking can be provided. Thereto, the handle piece 8 includes an unlocking mechanism, as it is described in more detail below. In the present embodiment, the unlocking mechanism is implemented by means of an unlocking device 18 of the handle piece 8 and the step-shaped guide course of the guide structure 15.

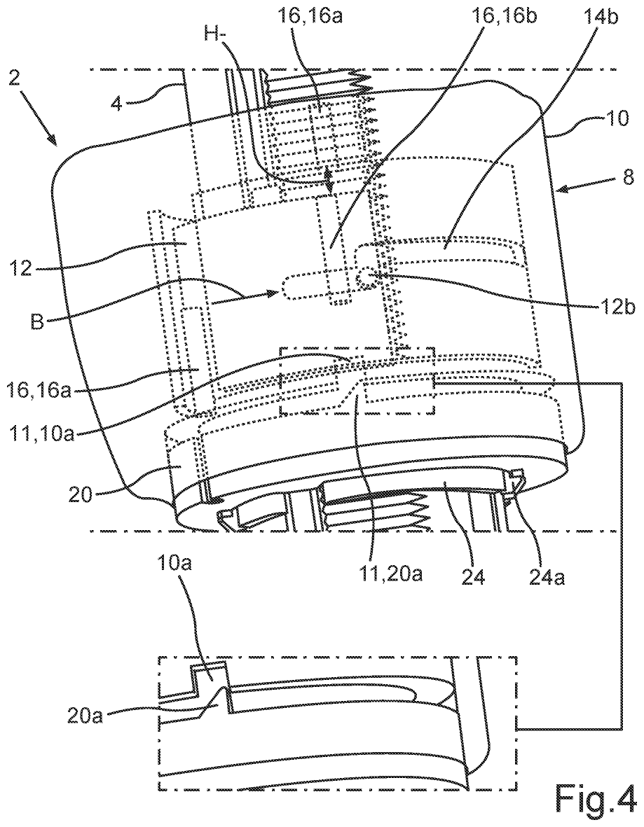

As shown in FIG. 4, sliding or pulling the adjusting element 10 along the longitudinal axis L, presently in particular upwards, is first provided for initiating the unlocking, to release the magnetic force of the fixing elements 16a, 16b. The movement direction and the movement margin are preset by the section of the guide rail 14b extending parallel to the longitudinal axis L.

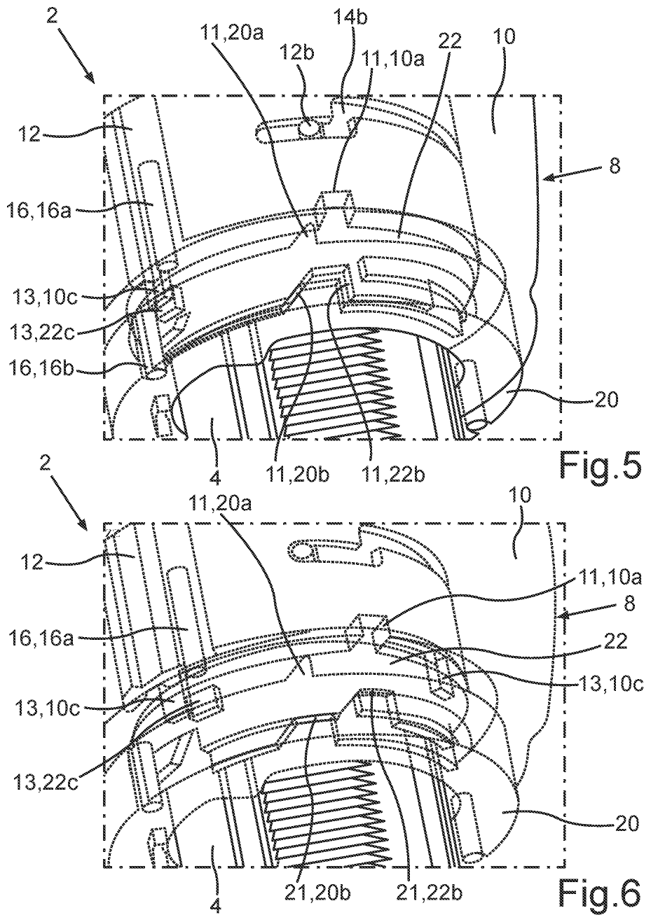

Subsequently, rotation of the adjusting element 10 further into the blocking direction B is provided, to perform the unlocking (see FIGS. 5 and 6). Correspondingly, the adjusting element 10 can be formed for releasing the fixing according to the unlocking mechanism to take an unlocking position different from the blocking position and the adjusting position by rotating from the blocking position into the blocking direction B. FIG. 6 shows the handle piece 8 in an unlocking state, in which the adjusting element 10 is in the unlocking position.

In the unlocking position, the first and the second fixing element 16a, 16b are moved into the above-mentioned unlocking pose. Therein, the respective rocker element 12 maintains the arresting pose in the unlocking position of the adjusting element 10. This means, the handle piece 8 is blocked, but no longer fixed or locked in the unlocking position.

In order to maintain the unlocking pose of the fixing elements 16a, 16b in rotating into the unlocking position or back into the adjusting position, the unlocking device 18 is provided. The unlocking device 18 is for example three-part constructed and arranged between the sliding part 4 and the adjusting element 10. In the embodiments, the unlocking device is arranged along the longitudinal axis below the respective rocker element 12.

The unlocking device 18 includes a first adjusting element 20, a second adjusting element 22 and a signaling element 24. The first unlocking element 20 is arranged between the adjusting element 10 and the sliding part 6. The second adjusting element 22 is overlapped or surrounded by the first unlocking element 20 at least in certain areas or in sections along the sliding direction T and arranged between the sliding part 4 and the adjusting element 10. The signaling element 24 is arranged between the sliding part 6 and the first and the second unlocking element 20, 22.

The first adjusting element 20 is non-rotatably arranged in relation to the sliding part 6. However, the adjusting element 10 is slidable along the longitudinal axis L in relation to the first unlocking element 20. The second unlocking element 22 is stationary against sliding along the longitudinal axis L in relation to the adjusting element 10. However, the second unlocking element 22 is rotatable, in particular rotatable at least in sections, into the rotational direction R in relation to the adjusting element 10. In contrast, the signaling element 24 is formed completely stationarily or non-rotatably to the adjusting element 10. The first adjusting element 20 is preferably slidable in relation to the signaling element 24 along the longitudinal axis L.

The first unlocking element 21 and the adjusting element 10 form at least one, presently for example two spacing structures 11 to each other. Upon rotating the adjusting element 10 transversely to the longitudinal axis outside of the blocking position, the adjusting element 10 and the first unlocking element 21 are maintained at a preset distance in relation to each other by means of the respective spacing structure 11. This means, the first unlocking element 20 and the adjusting element are supported against each other outside of the blocking position. Thereby, the unlocking pose of the fixing elements 16a, 16b is realized (see for example FIGS. 5 and 6).

The respective spacing structure 11 is presently formed by a supporting part and a retaining part 10a as the spacing body. The supporting part 20a extends along the longitudinal axis L out of the first unlocking element 20 as a projection or nose. The retaining part 10a is included by the adjusting element 10 and is formed by a resting surface or supporting surface. The resting surface extends axially along the inner side of the adjusting element 10 facing the sliding part 4 along the rotational direction R. For realizing the unlocking pose, the supporting part 20a rests on the resting surface of the retaining part 10a. Upon rotating, thus, the nose slides along the resting surface.

In order to still improve the fixing, the spacing structure 11 includes a locking mechanism. By means of it, the first unlocking element 20 and the adjusting element are connectable in form-fit manner in the blocking position, whereby the preset distance is reduced and the fixing pose of the fixing elements 16a, 16b is implemented. Presently, the described retaining part 10a includes a receptacle or recess corresponding to the projection of the supporting part 20a. This recess extends along the sliding direction T corresponding to the supporting part 20a. In the blocking position, the receptacle is formed to receive the nose of the supporting part 20a. The nose can latch into or engage with the receptacle. Thereby, the distance preset by the nose is reduced and the fixing elements 16a, 16b can be brought into the fixing pose.

The first unlocking element 20 is coupled or connected to a guide groove 24c with the signaling element 24 via a guide stud 20c. The guide groove 24c extends in the sliding direction T along the signaling element 24. Thus, the first unlocking element 20 is slidable in relation to the signaling element 24 along the longitudinal direction L.

In the received state, the guide groove 24c limits the movement of the first unlocking element 20 presently to the top. Thereby, by pulling on the adjusting element 10 for releasing the fixing, the nose can be guided out of the receptacle at least in certain areas, preferably completely (see FIG. 4). Therein, the retaining force of the fixing elements 16a, 16b releases itself at least as far as the magnetic force is no longer strong enough to retain the first unlocking element 20. The first unlocking element 20 thereby drops in the direction of gravity presently downwards towards the receiving part 6.

In the direction of the receiving part 6 of the apparatus 2, the signaling element 24 includes at least one, presently for example two, in particular for example three or more stops 24a. By means of the respective stop 24a, the first unlocking element 20 is supported from one side at least in the unlocking pose, thus outside of the blocking position. On the opposing side, the receiving part 6 can for example be supported with respect to the handle part 8 by means of the stop 24a.

Since the signaling element is formed stationary to the adjusting element 10 and the first unlocking element 20, the signaling element 24 can protrude beyond the adjusting element 10 along the longitudinal axis L (presently for example downwards) in the blocking position. The signaling element 24 is exposed in certain areas and thus visible by the operator. Thereby, blocking and in particular also fixing the handle part 8 by means of the signaling element 24 can be signaled (see FIG. 3). In particular, the signaling element 24 can for example be colored in a signal color such as for example red.

The second unlocking element 22 forms at least one, presently for example two sliding structures 21 with the first unlocking element 20. By means of the respective sliding structure 21, the adjusting element 10 and the first unlocking element 20 are slidable in relation to each other into the preset distance along the longitudinal axis L. This means, the unlocking structure 21 is employed to automatically realize the unlocking by rotating the adjusting element 10 into the unlocking position. Preferably, the sliding structure 21 can also be employed for maintaining the above-mentioned distance analogously to the spacing structure 11.

The respective sliding structure 21 is presently for example formed by two sliding bodies, namely a first sliding body 20b and a second sliding body 22b. The first sliding body 20b is arranged on the inner side of the first unlocking element 20 facing the second unlocking element 22. The first sliding body 20b is for example formed as a bulge or projection, which extends along the longitudinal direction L (presently for example upwards) towards the second unlocking element 22. The first sliding body 20b comprises a resting surface (presently the top side), by means of which the first sliding body 20b can be supported against the second sliding body 22b.

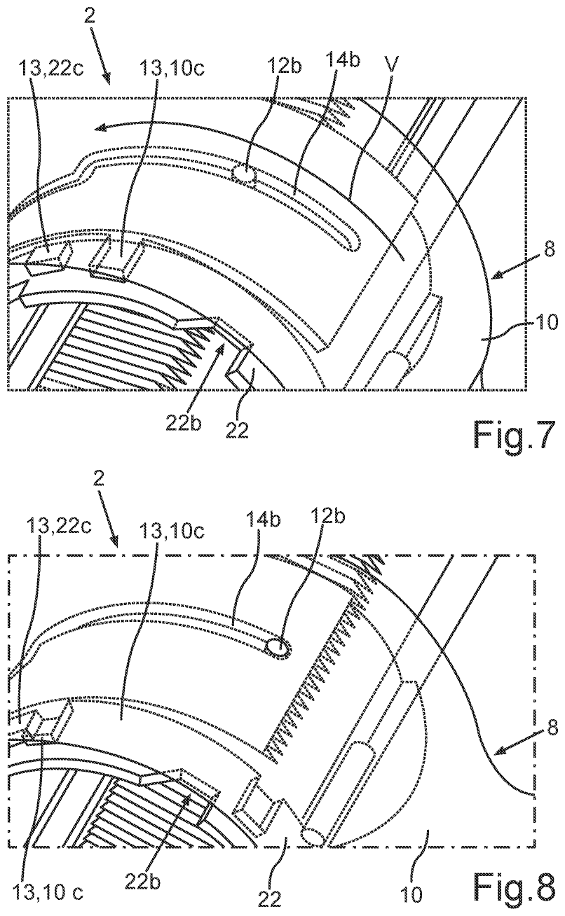

The second sliding body 22b is included by the second unlocking element 22. The second sliding body 22b includes a resting surface or supporting surface for the resting surface of the first sliding body 20b. As shown in FIGS. 7 and 8, the resting surface of the second sliding body 22b is formed by a lower edge area of the second unlocking element 22.

The second sliding body 22b additionally includes a receptacle or recess corresponding to the first sliding body 20b, in particular the bulge. The first sliding body 20b can be received in this receptacle or recess in the blocking position, as shown in FIG. 3. Thereby, the first sliding body 20b and the second sliding body 22b form a form-fit connection. As shown in FIG. 1, the recess and the projection have a rectangular shape, in which one side extends obliquely to the sliding direction T. This means, a ramp is formed. By this ramp, the projection can particularly simply slide along the recess upon rotating the adjusting element 10 from the blocking position into the blocking direction and thus the second unlocking element 22 and in particular the adjusting element 10 connected thereto and the first unlocking element 20 can be brought into the preset distance to each other (see FIGS. 6 and 7). As shown in FIG. 1, the unlocking device can include multiple, for example two of the previously described spacing structures and sliding structures.

Finally, the second unlocking element 22 and the adjusting element 10 form at least one, presently for example two coupling structures 13 to each other. By means of the respective coupling structure 13, the second unlocking element 22 can be carried along transversely to the longitudinal axis L at least in sections upon rotating the adjusting element 10. In particular, the second adjusting element 22 is only carried along upon rotating from the blocking position into the unlocking position and upon rotating the adjusting element 10 from the unlocking position into the adjusting position at least in sections.

Presently the respective coupling structure 13 is for example formed by two entraining bodies 11c and a respective carrying body 22c. The respective entraining body 11c is formed as a projection or block protruding radially to the longitudinal axis L on the inner side of the adjusting element 10 facing the second unlocking element 22. The respective carrying body 22c is formed as the outer side of the second unlocking element 22 protruding radially to the longitudinal axis L in the direction of the adjusting element 10. In the coupled state, the respective entraining body 11c applies the carrying body 22c with a force generated by rotating, by means of which the entraining body 22c is rotationally carried along with the carrying body 11c.

The two carrying bodies 11c are arranged offset along the rotational direction R. The entraining body 22c is arranged between the two carrying bodies 11c. As shown in FIGS. 5 and 6, the axial arrangement of the carrying bodies 11c is selected such that one of the carrying bodies 11c is coupled to the entraining body 22c in the blocking position. Upon rotating the adjusting element 10 into the blocking direction, thereby, the second unlocking element 22 is also rotated. Thereby, the sliding structure 21 is activated.

FIG. 7 and FIG. 8 show the return of the adjusting element 10 from the unlocking position back into the initial position. Thereto, FIG. 7 shows the handle piece 8 in a state, in which the adjusting element 10 is rotated by approximately two thirds starting from the unlocking position back into the adjusting position. For better clarity, only the second adjusting element 22 is illustrated of the unlocking device 18 to more clearly illustrate the function of the coupling structure 13.

In FIG. 7, the other one of the carrying bodies 11c as well as the entraining body 22 are just not yet in the coupled state. However, the coupling is effected before reaching the adjusting position. Thus, the second unlocking element 22 is only entrained in the last section of the guide rail 14b upon rotating into the adjusting direction V. Related to FIG. 6, thus, the distance between the first unlocking element 20 and the adjusting element 10 is maintained by the sliding structure 21, without the supporting part 20a and the notch of the retaining part 10a engaging with each other upon rotating and sliding back into the adjusting position. Only if the supporting body is again offset in relation to the receptacle, the second adjusting element 22 is carried along by the coupling structure 13 until the adjusting element 10 is again in the initial pose, thus in the adjusting position.

Overall, the embodiments show how a lockable twistlock, thus a lockable adjusting element 10, for example for a handle piece 8 of a medial handpiece, can be realized.

Although only a few embodiments of the present invention have been shown and described, it is to be understood that many changes and modifications may be made thereunto without departing from the spirit and scope of the invention.

### REFERENCE CHARACTERS

2 Apparatus
4 sliding part
4a tooth device
4b scale
4c bearing groove
6 receiving part
8 handle piece
10 adjusting element
10a retaining part
10c carrying body
11 spacing structure
12 rocker element
12a tooth device
12b guide pin
12c bearing stud
13 coupling structure
14 guide element
14b guide rail
15 guide structure
16 fixing device
16a first fixing element
16b second fixing element
18 unlocking device
20 first unlocking element
20a supporting part
20b sliding body
20c guide stud
21 sliding structure
22 second unlocking element
22b second sliding body
22c entraining body
24 signaling element
24a stop
24c guide groove
B blocking direction
L longitudinal axis
R rotational direction
T sliding direction

What is claimed is:

1. An apparatus for setting a penetration depth of a tubular or rod-shaped sliding part, in particular of a medical aspiration needle carrier part in a receiving part, the apparatus comprising a handle piece, which surrounds the sliding part, and which is formed for sliding along a longitudinal axis L of the sliding part in relation to the sliding part, and the handle piece comprising:
an adjusting element, which is formed for rotating transversely to the longitudinal axis L of the sliding part, and
at least one rocker element, which is arranged between the adjusting element and the sliding part, wherein
the adjusting element is formed to pivot the respective rocker element into an arresting pose to the sliding part, in which sliding of the handle piece along the longitudinal axis L is blocked, by rotating into a blocking direction B transversely to the longitudinal axis L into a blocking position, and
the adjusting element is formed to pivot the respective rocker element into a release pose to the sliding part, in which sliding of the handle piece along the longitudinal axis L is released, by rotating into an adjusting direction V different from the blocking direction B transversely to the longitudinal axis L into an adjusting position different from the blocking position, and further comprising
at least one fixing device with a first fixing element rotatable transversely to the longitudinal axis L to the sliding part by means of the adjusting element and a second fixing element stationary to the sliding part against the rotation transverse to the longitudinal axis L, wherein
the first fixing element and the second fixing element are formed to exert a retaining force H for fixing the adjusting element against the rotation transverse to the longitudinal axis L in the blocking position.

2. The apparatus according to claim 1, wherein the first fixing element forms a barrier body, and the second fixing element forms a notch configured for receiving the barrier body, wherein the barrier body is formed to be moved into a fixing pose, in which the retaining force (H) is maximum, by means of an operating action of an operator in the blocking position of the adjusting element.

3. The apparatus according to claim 1, wherein the adjusting element is formed to move the first fixing element into a fixing pose to the second fixing element, in which the retaining force H is maximum, by rotating transversely to the longitudinal axis L into the blocking position.

4. The apparatus according to claim 3, wherein the first fixing element and the second fixing element include a magnetic material and form a magnetic field of opposite polarity to each other.

5. The apparatus according to claim 3, wherein the first fixing element is formed as a rolling bearing with at least one rolling body, and the second fixing element forms a notch for receiving the rolling body in the fixing pose.

6. The apparatus according to claim 1, wherein the adjusting element and the respective rocker element form a guide structure, which presets a guide course at least for rotating the adjusting element transversely to the longitudinal axis L in relation to the respective rocker element, wherein the adjusting element is adjustable into the respective position along the guide course.

7. The apparatus according to claim 6, wherein the guide structure includes a guide course section associated with the blocking position, which extends along the longitudinal axis L, and the adjusting element is slidable along the guide course section in relation to the respective rocker element for releasing the fixing.

8. The apparatus according to claim 3, wherein the adjusting element is formed to occupy an unlocking position different from the blocking position and the adjusting position by rotating from the blocking position into the blocking direction B for releasing the fixing, and thereby to move the first fixing element and the second fixing element into an unlocking pose, in which the retaining force H is reduced compared to the fixing pose, wherein the respective rocker element maintains the arresting pose in the unlocking position of the adjusting element.

9. The apparatus according to claim 6, wherein the adjusting element is formed to occupy an unlocking position different from the blocking position and the adjusting position by rotating from the blocking position into the blocking direction B for releasing the fixing, and thereby to move the first and the second fixing element into an unlocking pose, in which the retaining force H is reduced compared to the fixing pose, wherein the respective rocker element maintains the arresting pose in the unlocking position of the adjusting element.

10. The apparatus according to claim 1, wherein the handle piece includes an unlocking device, which is arranged between the sliding part and the adjusting element, said unlocking device being formed to maintain the respective fixing elements in the unlocking pose upon rotating the adjusting element transversely to the longitudinal axis.

11. The apparatus according to claim 10, wherein the unlocking device includes a first unlocking element, which is stationarily arranged to the sliding part against the rotation transverse to the longitudinal axis, and the adjusting element and the first unlocking element form a spacing structure, wherein the spacing structure is formed to maintain the adjusting element and the first unlocking element at a preset distance in relation to each other along the longitudinal axis L upon rotating the adjusting element transversely to the longitudinal axis L, and thereby to realize the unlocking pose of the fixing elements, and wherein the spacing structure includes a connection mechanism, by means of which the first unlocking element and the adjusting element are connected in form-fit manner in the blocking position and therein the preset distance is reduced.

12. The apparatus according to claim 10, wherein the unlocking device includes a signaling element, which protrudes beyond the adjusting element at least in certain areas along the longitudinal axis, the signaling element being configured for signaling the blocking and fixing by sliding the first unlocking element only in the blocking position.

13. The apparatus according to claim 10, wherein the unlocking device includes a second unlocking element, which is stationarily arranged to the adjusting element against sliding along the longitudinal axis L, wherein the first and the second unlocking element form a sliding structure, wherein the sliding structure is formed to slide the adjusting element and the first unlocking element into the preset distance to each other in relation to each other along the longitudinal axis L upon rotating the adjusting element transversely to the longitudinal axis L out of the blocking position into the unlocking position.

14. The apparatus according to claim 10, wherein the adjusting element and a second unlocking element form a coupling structure for carrying along the second unlocking element at least in sections upon rotating the adjusting element transversely to the longitudinal axis L, wherein the coupling structure is formed to offset the spacing structure and the sliding structure transversely to the longitudinal axis L upon sliding the adjusting element from the unlocking position into the adjusting position and thereby to maintain the preset distance upon rotating into the adjusting direction V.

15. The apparatus according to claim 1, wherein the first fixing element is embraced by the adjusting element and the holding force H must be overcome to release the fixing.

16. A medical handpiece for a medical appliance, with a first and a second apparatus according to any one of the preceding claims, wherein the first apparatus is formed to set the penetration depth of a first tubular sliding part in a first receiving part, and wherein the second apparatus is formed to set the penetration depth of a second tubular sliding part in a second receiving part, and the first apparatus and the second apparatus are arranged one behind the other such that the first sliding part of the first apparatus is at least partially formed as a second receiving part for the second sliding part of the second apparatus, wherein the first receiving part and the second sliding part comprise a respective connection interface for connecting elements of the medical appliance.
